(19) United States
(12) Patent Application Publication
(10) Pub. No.: US 2022/0008117 A1
(43) Pub. Date: Jan. 13, 2022

(54) RADIOFREQUENCY ELECTRODE FOR USE IN A SURGICAL HANDHELD DEVICE, ELECTRODE INSTRUMENT AND RESECTOSCOPE

(71) Applicant: OLYMPUS WINTER & IBE GMBH, Hamburg (DE)

(72) Inventors: Alexander CARROUX, Barsbüttel (DE); Thomas WOSNITZA, Lüneburg (DE)

(73) Assignee: OLYMPUS WINTER & IBE GMBH, Hamburg (DE)

(21) Appl. No.: 17/369,645

(22) Filed: Jul. 7, 2021

(30) Foreign Application Priority Data
Jul. 7, 2020 (DE) 10 2020 117 810.7

(51) Int. Cl. A61B 18/14

(52) U.S. Cl. CPC A61B 18/149; A61B 2018/00077

(57) ABSTRACT

A radiofrequency electrode for handheld devices are used predominantly in urology for electrosurgical work in the bladder, the prostate, and the urethra. A large active area of the electrode is known to be advantageous. However, the size of the area is restricted by the available space. Moreover, greater energy is required for igniting the plasma for large electrodes. However, an elevated energy level is disadvantageous in terms of the heat influx into the rinsing liquid and this may be disadvantageous for the surrounding tissue. The invention develops a radiofrequency electrode, an electrode instrument and a resectoscope, in which a plasma can be better localized at the electrode and the energy for igniting the plasma is reduced at the same time. This is achieved by virtue of a radiofrequency electrode for use in a surgical handheld device having the shape of a toroid.

The invention relates to a radiofrequency electrode for use in a surgical handheld device as per the preamble of claim 1. Moreover, the invention relates to an electrode instrument for use in a surgical handheld device as claimed in claim 13 and to a resectoscope as claimed in claim 14.

Generic radiofrequency electrodes for handheld devices, more particularly resectoscopes, are used predominantly in urology for electrosurgical work in the bladder, prostate, and the urethra. Equally, these radiofrequency electrodes can also be used for other surgical and orthopedic treatments or interventions. As a rule, these electrodes are used for resection and vaporization and also electrocoagulation of tissue, e.g., of tissue in the lower urinary tract. To this end, the handheld devices or the resectoscopes comprise a generic radiofrequency electrode or an electrode instrument, which is mounted within a shaft of the handheld device or the resectoscope so as to be longitudinally displaceable and rotatable. The surgical radiofrequency electrode is arranged at a distal working end of the electrode instrument, for example in the shape of a loop or a button.

Such electrodes can have a monopolar or bipolar embodiment. Depending on application or embodiment, electric power or a radiofrequency voltage is applied to the electrodes to this end via electrical conductors. The supply with the radiofrequency current is implemented via the handheld device or the resectoscope by means of a radiofrequency generator. A neutral electrode is arranged on the person to be treated in the case where the electrode is embodied as a monopolar electrode. Alternatively, the neutral electrode can also be a constituent part of the resectoscope. In this case, the shaft, the carrier, and the optical unit serve as neutral electrode of an electric potential.

A plasma of electrically charged particles forms around the electrode or around some parts of the electrode as a result of applying a conventional radiofrequency voltage to the electrode. The plasma is localized directly at the electrode and has a high temperature or energy density, which is why it is particularly well-suited to the aforementioned purposes. For vaporization and electrocoagulation of tissue in particular, it is essential for the plasma to be generated at a certain area of the electrode so that the treating person can use the electrode with pinpoint accuracy.

A plasma which tends to be oriented toward the electrical conductors of the electrode sometimes forms in known radiofrequency electrodes. This prevents targeted use of the electrode and the plasma density at the electrode itself is reduced. Ideally, the plasma forms at a lower or a mid region of the electrode where the electrode is in contact with the tissue. The formation of the plasma in an upper region of the electrode, where the electrical conductors are situated, was found to be inexpedient for surgical use.

The electrode having an active area that is as large as possible is advantageous for the aforementioned treatments. However, firstly, the size of the area is restricted by the dimension of the region of use and, secondly, a higher energy level is required to ignite the plasma for large electrodes. However, increased heat has a disadvantageous effect on the region in the body to be treated. Moreover, a larger electrode reduces the ignition speed.

The invention is based on the object of developing a radiofrequency electrode, an electrode instrument and a resectoscope in which the plasma can be better localized at the electrode, as a result of which the area which is in tissue contact is maximized and the energy level for igniting the plasma is reduced at the same time.

A solution to this object is described by the features of claim 1. Accordingly, provision is made for the radiofrequency electrode for use in a surgical handheld device, more particularly a resectoscope, to have the shape of a toroid. This toroidal or donut-like electrode has an outer circumference and an inner circumference. In this case, the electrode body describes a closed ring structure. To use this electrode, for example for vaporization, for resection or for electrocoagulation, the lower half of the toroid is moved over the tissue of a patient to be treated. The toroidal shape leads to this radiofrequency electrode having a large active area which, on the other hand, is reduced in terms of its area by way of the central opening. This reduced active surface leads to a lower electric energy level being required to ignite the plasma than in the case of known full-face electrodes. This also allows the ignition speed to be increased or the ignition time to be reduced. A further advantage of the toroidal or donut-like electrode should be considered to be the fact that bubbles in the rinsing liquid, which arise due to the plasma and which impair the view, can be removed in controlled fashion through the hole in the electrode. The view in front of the optical unit can be improved by this removal of the bubbles.

Preferably, provision can be made for the toroidal electrode for generating a plasma to be connected to an electrical conductor, by means of which the electrode is able to be coupled to a radiofrequency generator. The electrical conductor is coupled to a lower region or a lower half of the toroid in the case of this electrode. This lower region is electrically conductive. The complementary upper region or the upper half of the electrode can have an electrically conductive or electrically insulating embodiment. As a result, the electric power is deposited precisely where the plasma should be generated. In this case, for example, the electrical conductor can be guided through a shaft of the handheld device, more particularly of the resectoscope, to the radiofrequency generator. In this case, the electrical conductor can be connected both to the radiofrequency electrode and to the handheld device in integral fashion, or it can be able to be coupled in plug-like fashion to the handheld device and/or the radiofrequency electrode. Thus, it is conceivable for the radiofrequency electrode to have one or two conductors, by means of which the electrode is able to be connected to the handheld device in plug-like fashion. This is particularly advantageous, in particular for maintenance and cleaning purposes.

In particular, the preliminary invention alternatively provides for the toroidal electrode for generating a plasma to comprise two electrical conductors, said electrical conductors each contacting an electrical pole of the electrode and each being able to be coupled to a radiofrequency generator, wherein the electrical poles of the electrodes are electrically insulated from one another by way of an insulator element. The electrical conductors each lead to a pole at the electrode in the case of this bipolar electrode. In this case the radiofrequency electrode is subdivided into two poles. Preferably, one pole is positioned at a lower side and the other pole is positioned at an upper side of the electrode. The insulator element is located between the poles. It is also however conceivable for the poles on the electrode to be divided differently. Thus, it is conceivable for a pole on the lower side of the electrode to have a substantially larger embodiment than the second pole, which only extends in ring-like fashion on the upper side of the electrode. As a result of this special configuration of the electrode, the plasma provided for surgical use can be localized particularly well and can consequently be used with pinpoint accuracy. Moreover, it is conceivable for the other or the second pole, or the neutral electrode, to be arranged on one of the two support arms of the electrode instrument. Thus, it is conceivable that the two support arms or fork tubes of the electrode can also serve as return electrode and/or neutral electrode.

Moreover, provision can be made according to the invention for the electrode to be fastened, preferably detachably fastened, to the handheld device, in particular to an electrode instrument, via the at least one electrical conductor and/or via at least one holding element. In addition to the at least one electrical conductor, a further element made of, e.g., a metal or an insulator can consequently ensure the stability of the electrode on the handheld device or on the electrode instrument. Elevated mechanical tensile and compressive forces act on the electrode, in particular while the electrode is used. The holding element and the at least one electrical conductor prevent the electrode from losing its position relative to the handheld device even in the case of elevated mechanical forces. The holding element can also have a plug-like embodiment such that a detachable connection or a coupling connection with the handheld device is facilitated.

A further exemplary embodiment of the invention can provide for the at least one electrical conductor, preferably two electrical conductors, to contact the toroid at an inner side. The conductors do not interfere during the treatment as a result of this positioning of the conductors at the inner circumference of the toroid. As a result of the electrical conductors being assigned to the inner circumference, it is possible to use the outer circumference of the toroid and the lower side for the purposes of cutting or vaporizing the tissue.

Preferably, it is further conceivable for a cross section parallel to a radial axis of the toroid to have an embodiment that is elliptical, oval, circular, triangular, quadrilateral, preferably square, trapezoidal, polygonal or the like. Different plasma densities at the surface of the toroid can be generated by these different cross sections. Shapes with acute angles in particular allow the generation of particularly high plasma density, which is advantageous for certain uses, for example tissue being cut. Equally, tissue regions can be vaporized or coagulated particularly well by way of large areas.

Further, it is conceivable for the outer circumference of the toroid to have an elliptical, oval or circular embodiment. The use for certain surgical methods can also be optimized by the shape of the circumference. Depending on the region to be treated and the type of treatment, it may be advantageous to use an elliptical, oval or circular toroid.

A special exemplary embodiment of the present invention can provide for a ring-like protrusion, preferably an edge, to be formed around an outer circumference of the toroid surface. This protrusion promotes the ignition of the plasma or implements ignition particularly quickly, to be precise with less energy outlay. The plasma ignites quickly due to the high electric field strength at this ring-like protrusion, as a result of which the electrode can be used particularly quickly. It is also conceivable for this ring-like protrusion to be formed as an edge only in regions or to be positioned at a different position on the surface of the toroid.

Further, it is conceivable that a first electrical conductor is assigned to a lower section of the toroid and this section forms a first electrical pole and a second electrical conductor is assigned to an upper section of the toroid and this section forms a second electrical pole, wherein the lower and the upper section have the same or a different embodiment in terms of area. In particular, the lower section is larger or smaller than the upper section in terms of area.

Further, it is conceivable that an outer diameter, in particular mean outer diameter, of the toroid is 2 to 5 times, more particularly 2.5 to 4, or 3 times larger than an inner diameter, in particular mean inner diameter, of the toroid. Moreover, further ratios of the diameters are also conceivable. Thus, provision can also be made for the toroid to have a virtually ring-like embodiment or for it to be almost completely closed. Certain geometries can be particularly advantageous depending on use.

The toroid substantially consists of stainless steel, titanium, platinum iridium or platinum tungsten. Moreover, it is conceivable for the toroid to also be constructed from a different electrically conductive material. The electrical insulator can be formed from a plastic or a ceramic.

An electrode instrument for achieving the stated object has the features of claim 13. Accordingly, provision is made for the electrode instrument for use in a surgical handheld device, more particularly in a resectoscope, to comprise an elongate shaft section with two support arms, through which at least one conductor extends, the latter being connected or being able to be connected to a radiofrequency electrode as claimed in claims 1 to 12 at the distal end of the instrument. The radiofrequency electrode is arranged between the distal ends of the support arms.

A resectoscope for achieving the object stated at the outset has the features of claim 14.

Preferred exemplary embodiments of the invention will be described in more detail below with reference to the drawing. In this drawing:

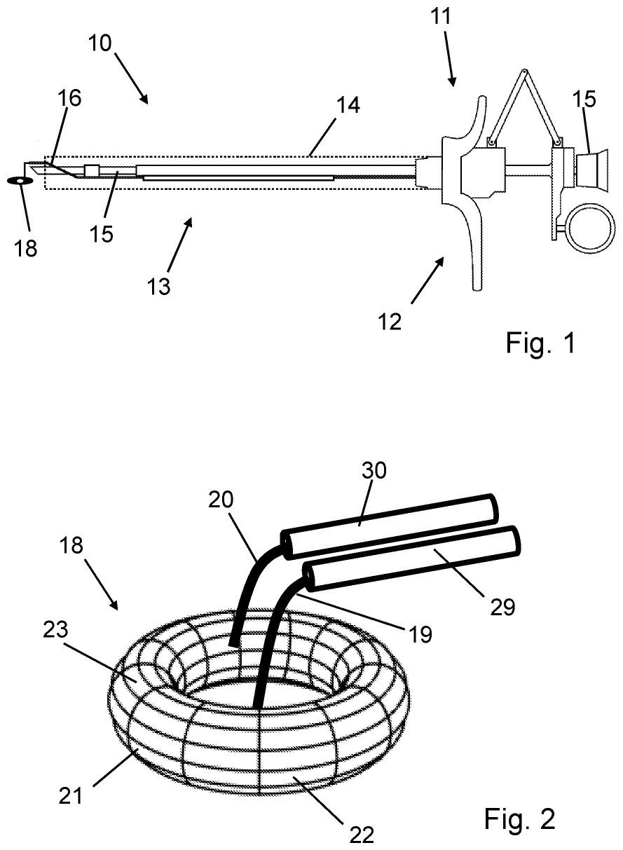

FIG. 1 shows a schematic illustration of a surgical handheld device, more particularly a resectoscope,

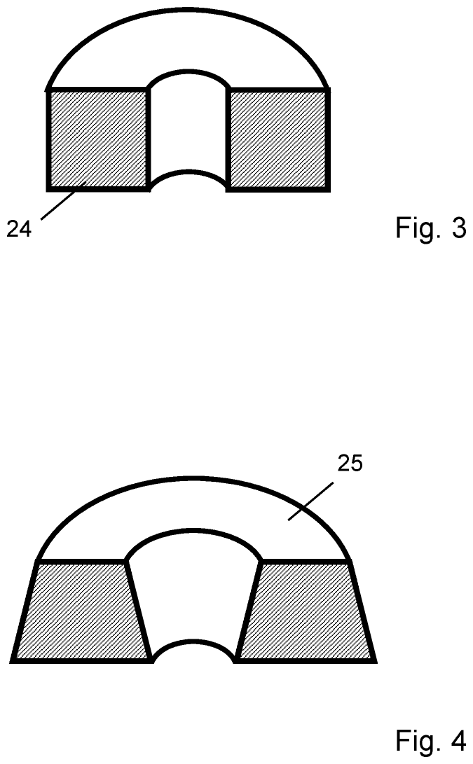

FIG. 2 shows an exemplary embodiment of a radiofrequency electrode,

FIG. 3 shows a further exemplary embodiment of a radiofrequency electrode,

FIG. 4 shows a further exemplary embodiment of a radiofrequency electrode,

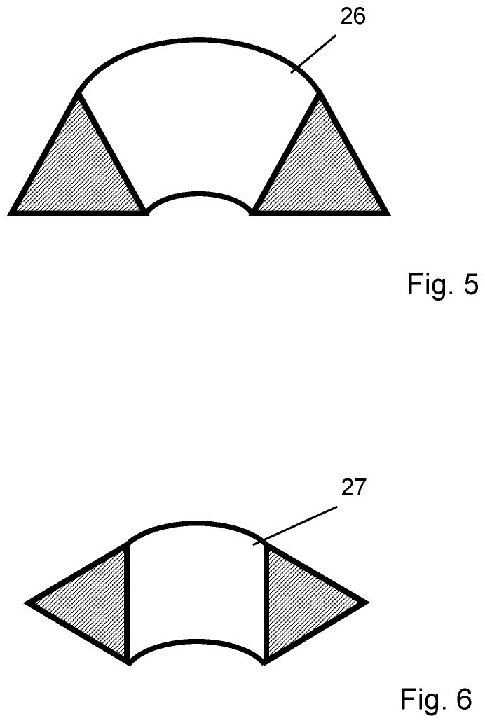

FIG. 5 shows a further exemplary embodiment of a radiofrequency electrode,

FIG. 6 shows a further exemplary embodiment of a radiofrequency electrode, and

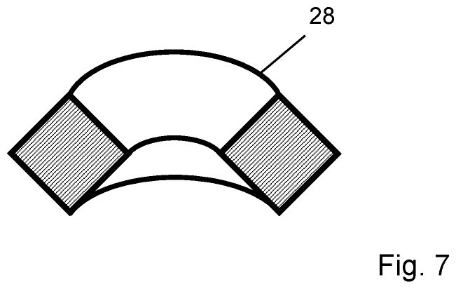

FIG. 7 shows a further exemplary embodiment of a radiofrequency electrode.

FIG. 1 illustrates a resectoscope 10 as an example of a surgical handheld device. This resectoscope 10 substantially consists of a carrier 11, a grip unit 12 and a shaft 13, which should be guided into an appropriate body orifice for the purposes of treating a patient. In the exemplary embodiment illustrated here, the shaft 13 is composed of an outer shaft tube 14, an optical unit 15 and an electrode instrument 16. The optical unit 15 consists of a long tube in which lenses or optical fibers can be arranged in order to observe the region of the treatment at the distal end of the shaft 13 through an eyepiece 17 arranged proximally on the shaft 13. Reference is made to the known prior art in respect of a more detailed description of a resectoscope.

The electrode instrument 16 is substantially composed of a radiofrequency electrode 18 and at least one electrical conductor 19. Firstly, the at least one electrical conductor 19 supplies the radiofrequency electrode 18 with a radiofrequency voltage and, secondly, the conductor 19 and optionally a further element serve as a holder of the electrode 18 on the electrode instrument 16. The electrical conductor 19 runs from the distal end of the resectoscope 10 through the shaft 14 and is connected by further lines to a radiofrequency generator (not illustrated) for generating the radiofrequency electromagnetic energy. The two conductors 19, 20 are able to be guided through the two support arms 29, 30 or through the fork tubes of the electrode instrument 16 (FIG. 2).

Tissue, for example, can be manipulated by means of the radiofrequency electrode 18. To this end, the radiofrequency electrode 18 can be embodied either as a monopolar electrode or as a bipolar electrode. In the case of a bipolar electrode, the latter is connected to two electrical conductors 19, 20. In the exemplary embodiment of a monopolar electrode, the electrode 18 is only connected to one electrical conductor 19. A further neutral electrode is attached to the patient or is integrated in the resectoscope (shaft, carrier, optical unit; if all components are at one electrical potential, this leads to a lower current density on account of the large area). A plasma by means of which tissue is manipulated by way of an appropriate movement of the electrode instrument 16 is generated at the electrode 18 by supplying the radiofrequency electrode 18 with electric power.

The radiofrequency electrode 18 as per the present invention is embodied as a toroid 21. In FIG. 2, a toroid 21 with a circular cross section is illustrated in exemplary fashion. This toroid 21 is electrically connected to the power source by way of two electrical conductors 19, 20 of the electrode instrument 16. Alternatively, it is conceivable for one of the conductors 19, 20 to be embodied as a holding element.

The toroid 21 or the radiofrequency electrode 18 has a lower region 22 and an upper region 23. These regions 22, 23 can have different sizes. In the exemplary embodiment illustrated here, the electrical conductor 19 leads to the lower region 22 and the electrical conductor 20 leads to the upper region 23 of the toroid 21. Equally, it is conceivable for the upper region 23 to be affixed to by the further holder. An insulator for insulating the two electric poles from one another is provided between the electrically conductive regions 22 and 23 in the case of a bipolar electrode. A particularly large plasma in terms of area, which allows much tissue to be ablated within a short period of time, can be generated at the lower region 22 of the electrode 18 by this special embodiment of the radiofrequency electrode 18. The active surface of the electrode 18 can be kept small by the toroidal shape of the radiofrequency electrode 18, and so a small amount of heat or little electric energy is required for igniting the plasma. Moreover, this shape is particularly advantageous for fast ignition of the plasma.

The ratio between an outer circumference of the “donut-shaped” radiofrequency electrode 18 and an inner circumference can assume any desired values. Depending on the type of treatment or the use, it is possible to use toroids 21 with different dimensions. FIGS. 3 to 7 illustrate different toroidal structures. Thus, FIG. 3 shows an example of a radiofrequency electrode 18, in which a cross section of a toroid 24 has a rectangular or square embodiment. In the toroid 25 illustrated in FIG. 4, the cross section has a trapezoidal embodiment. The exemplary embodiment in FIG. 5 shows a toroid 26 with a triangular cross section. The exemplary embodiment of a toroid 27 as per FIG. 6 likewise exhibits a triangular cross section. However, the triangle is tilted in such a way in this exemplary embodiment that one tip points to the outside. FIG. 7 illustrates a further exemplary embodiment of a toroid 28. As a result of the different shapes, different plasmas form at the radiofrequency electrode 18 on account of the different electric field strengths. Thus, a particularly advantageously shaped electrode 18 can be selected according to requirements. Here, explicit reference is made to the fact that the selection of toroidal shapes illustrated here is not exhaustive. Rather, it is conceivable for the toroids to be able to adopt virtually any shape.

One exemplary embodiment of the invention, not illustrated, can provide for an edge or ring-like fringe to be arranged at an outer side of the toroid 21. This edge promotes the formation of a plasma, as a result of which, firstly, the ignition energy is able to be reduced and, secondly, the ignition time can be shortened.

The radiofrequency electrode 18 according to the invention can be connectable to the resectoscope 10 in plug-like fashion together with the electrode instrument 16. Equally, it is conceivable for the electrode 18 to be connectable to the electrode instrument 16 in plug-like fashion. This is particularly advantageous for maintenance and cleaning purposes in particular.

Express reference is made to the fact that the use of the radiofrequency electrode 18 is not restricted to a urological application. Rather, it is also conceivable that this electrode 18 can find use in orthopedic or other surgical or medical uses.

1. A radiofrequency electrode for use in a surgical handheld device, more particularly in a resectoscope, wherein the radiofrequency electrode is able to be supplied with electric power by way of at least one electrical conductor, wherein the radiofrequency electrode has the shape of a toroid.

2. The radiofrequency electrode as claimed in claim 1, wherein the toroidal electrode for generating a plasma comprises an electrical conductor, by means of which electrical conductor the electrode is able to be coupled to a radiofrequency generator.

3. The radiofrequency electrode as claimed in claim 1, wherein the toroidal electrode for generating a plasma comprises two electrical conductors, said electrical conductors each contacting an electrical pole of the electrode and being able to be coupled to a radiofrequency generator, wherein the electrical poles of the electrode are electrically insulated from one another by way of an insulator element.

4. The radiofrequency electrode as claimed in claim 1, wherein at least one electrical pole, more particularly a neutral electrode and/or a return electrode, of the electrode is formed by a support arm or a fork tube of the electrode.

5. The radiofrequency electrode as claimed in claim 1, wherein the electrode is fastened, preferably detachably fastened, to the handheld device, in particular to an electrode instrument, via the at least one electrical conductor and/or at least on a holding element.

6. The radiofrequency electrode as claimed in claim 1, wherein the at least one electrical conductor, preferably the two electrical conductors, contact the toroid at an inner side.

7. The radiofrequency electrode as claimed in claim 1, wherein a cross section parallel to a radial axis of the toroid has an embodiment that is elliptical, oval, circular, triangular, quadrilateral, preferably square, trapezoidal, polygonal or the like.

8. The radiofrequency electrode as claimed in claim 1, wherein an outer circumference of the toroid has an embodiment that is elliptical, oval or circular.

9. The radiofrequency electrode as claimed in claim 1, wherein a ring-like protrusion, preferably an edge, is formed around an external circumference of the toroid surface.

10. The radiofrequency electrode as claimed in claim 3, wherein a first electrical conductor is assigned to a lower section of the toroid and this section forms a first electrical pole and a second electrical conductor is assigned to an upper section of the toroid and this section forms a second electrical pole, wherein the lower and the upper section have the same or a different embodiment in terms of area, in particular wherein the lower section is larger or smaller than the upper section in terms of area.

11. The radiofrequency electrode as claimed in claim 1, wherein an outer diameter, in particular mean outer diameter, of the toroid is 2 to 5 times, more particularly 2.5 to 4, or 3 times larger than an inner diameter, in particular mean inner diameter, of the toroid.

12. The radiofrequency electrode as claimed in claim 1, wherein the toroid substantially consists of stainless steel, titanium, platinum iridium or platinum tungsten and an electrical insulator consists of a ceramic or a plastic.

13. An electrode instrument, more particularly a monopolar or bipolar electrode instrument, for use in a surgical handheld device, more particularly in a resectoscope, wherein the electrode instrument comprises an elongate shaft section with two support arms, through which at least one conductor extends, the latter forming a radiofrequency electrode as claimed in claim 1, which is able to be impinged with a radiofrequency current, at the distal end of the electrode instrument, said radiofrequency electrode being arranged between the distal ends of the support arms.

14. A resectoscope comprising an electrode instrument as claimed in claim 13.
